(19) United States
(12) Patent Application Publication
(10) Pub. No.: US 2012/0003148 A1
(43) Pub. Date: Jan. 5, 2012

(54) RADIOIMMUNOTHERAPY AND IMAGING OF TUMOR CELLS THAT EXPRESS VIRAL ANTIGENS

(76) Inventors: Ekaterina Dadachova, Mahopac, NY (US); Arturo Casadevall, New Rochelle, NY (US); Howard Strickler, New York, NY (US); Robert D. Burk, New York, NY (US)

(21) Appl. No.: 12/448,235

(22) PCT Filed: Dec. 13, 2007
(86) PCT No.: PCT/US2007/025491

§ 371 Date: Dec. 10, 2009

Related U.S. Application Data
(60) Provisional application No. 60/876,052, filed on Dec. 19, 2006.

(51) Int. Cl. A61K 51/10; A61P 35/02; A61P 35/00

(52) U.S. Cl. 424/1.49

(57) ABSTRACT

Methods and compositions are provided for treating and imaging a virus-associated cancer, where the methods comprise administering to a subject a radiolabeled binding molecule, wherein the binding molecule binds to a virus antigen expressed by virus-associated cancer cells in the subject.

### CROSS-REFERENCE TO RELATED APPLICATIONS

This application claims the benefit of U.S. Provisional Patent Application No. 60/876,052, filed on Dec. 19, 2006, the content of which is hereby incorporated by reference.

### STATEMENT OF GOVERNMENT SUPPORT

The invention disclosed herein was made with U.S. Government support under Grants AI60507, AI33774, AI33142, AI52733, HL59842, U54 AI157158, CA078527 and AI51519 awarded by The National Institutes of Health. Accordingly, the U.S. Government has certain rights in this invention.

### FIELD OF THE INVENTION

The invention relates to radioimmunological methods of treating and imaging tumor cells expressing viral antigens with anti-virus antigen binding molecules, such as antibodies, bearing a radioactive isotope.

### BACKGROUND OF THE INVENTION

Radioimmunotherapy (RIT) was developed for treatment of cancer nearly three decades ago. The first clinical trials of RIT for hepatoma in the US were performed by Order et al. in the mid-1980s (Order et al. (1985) J. Clin. Oncol. 3:1573-82). RIT takes advantage of the specificity of the antigen-antibody interaction to deliver radionuclides that emanate lethal doses of cytotoxic radiation to tumor cells, and provides a valuable alternative to chemotherapy and external beam radiation therapy (EBRT) (Sharkey et al. (2005) J. Nucl. Med. 46 Suppl 1:115S-127S; Milenic et al. (2004) Nat. Rev. Drug Discov. 3:488-99).

RIT has been developed into a successful therapy for certain cancers as evidenced by the recent approval of monoclonal antibody (mAb) therapy-based drugs such as Zevalin® and Bexxar® (anti-CD20 mAbs labeled with 90-Y and 131-I, respectively) for the treatment of relapsed or refractory B-cell non-Hodgkin's lymphoma. Recent reports on the use of MT as an initial treatment for follicular lymphoma are encouraging, thus potentially making RIT first line therapy in some types of cancer (Kaminski et al. (2005) N. Engl. J. Med. 352:441-9). RIT has recently also been demonstrated for treating fungal and bacterial diseases (reviewed by Dadachova and Casadevall (2006) Q. J. Nucl. Med. Mol. Imaging 50:193-204).

There exists a need for improved methods of imaging and treating tumors. For example, in the USA in 2004 there were approximately 28,000 new cases of oral cavity and pharynx cancer, 10,500 new cases of cervical cancer and 8,000 new cases of Kaposi sarcoma. Oral and cervical cancers are associated with human papilloma virus, and Kaposi sarcoma is caused by Herpes Virus 8.

Conventional methods including chemotherapy and surgery have been recently supplemented by immunotherapeutic therapies. Currently existing radioimmunotherapy of cancer utilizes tumor-associated antigens that are “self” antigens in a patient's body, which results in significant uptake of the antibody in normal organs which might lead to toxicity. One challenge with immunotherapy is identification of antigens that are specific to the tumor such that an effect can be exerted while limiting damage to normal tissues. A monoclonal antibody (Rituximab) directed against the B-cell surface antigen, CD20, is increasingly used as a therapy for B-cell lymphomas. However, CD20 is expressed on all normal mature B cells and hence is not a specific tumor target.

Certain animal cancerous cells, including tumors, exhibit viral antigens both internally and on their surfaces or can be caused to exhibit such viral antigens. As a result, viral antigens present a valuable way of specifically targeting binding molecules to tumor cells. The invention described herein provides use of that specific targeting.

### SUMMARY OF THE INVENTION

The invention provides methods of treating a subject having a virus-associated cancer comprising administering to the subject a radiolabeled binding molecule, wherein the binding molecule binds to a virus antigen in and/or on virus-associated cancer cells in the subject.

The invention also provides methods of imaging and/or treating virus-associated tumor cells in a subject comprising administering to the subject a radiolabeled binding molecule, wherein the binding molecule binds to a virus antigen in and/or on virus-associated tumor cells in the subject.

In contrast to “traditional” radioimmunotherapy, virally-transformed cancer cells are antigenically very different from host tissue thus providing for very specific antigen-binding molecule interactions with the cancer cells. The present invention thus limits cross-reaction of radiolabeled binding molecules with host tissues, which is expected to result in less toxicity to normal organs than conventional RIT or chemotherapy.

### BRIEF DESCRIPTION OF THE FIGURES

FIG. 1. Binding of ¹⁸⁸Re-labeled HPV16 E6-specific mAb C1P5 and isotype-matching control mAb 18B7 (IgG1) to whole HPV16-expressing human cervical carcinoma CasKi cells. Rhombus—mAb C1P5; triangle—mAb 18B7.

FIG. 2A-2B. Scintigraphic images of CasKi tumor-bearing mice 24 hours post-injection with A) ¹⁸⁸Re-C1P5 mAb; B) ⁸⁸Re-18B7 mAb.

FIG. 3A-3F. Expression of E6 and E7 in human cervical carcinoma cell lines and of HBx in hepatocellular carcinoma cell line by Western blot and effect of MG132 proteasome inhibitor treatment on the levels of E6, E7 and HBx in these cell lines: A) E6 from protein extracts of CasKi cells treated with MG132 for 3 hrs; B) E7 from protein extracts of CasKi cells treated with MG132 for 3 hrs; C) E6 from protein extracts of CasKi cells treated with MG132 for 6 hrs; D) E7 from protein extracts of SiHa cells treated with MG132 for 3 hrs; E) E7 from protein extracts of HeLa S3 cells treated with MG132 for 3 hrs; F) HBX from protein extracts of Hep 3B2.1-7 cells treated with MG132 for 3 hrs.

FIG. 4A-4D. Detection of viral antigens in tumor cells and in tumors: A, B) immunofluorescence of fixed and permeabilized tumor cells. Left panels show light microscopy images of the cells. Heavily damaged cells are marked with arrows. Right panels show immunofluorescent images of the same slides treated with viral protein-specific mAbs followed by FTIC-conjugated polyclonal antibody to mouse IgGs: A—CasKi cells and E6-specific C1P5 mAb, B—Hep 3B2.1-7 cells and HBx-specific 4H9 mAb; C) immunohistochemistry of CasKi tumors. Left panel shows binding of E6-specific mAb C1P5. Right panel shows absence of binding of control mAb 18B7; D) western blot of Hep 3B2.1-7 tumor with HBx-specific mAb 4H9.

FIG. 5A-5D. Scintigraphic images of tumor-bearing mice 24 hours post-injection with: A) CasKi tumor, E6-specific mAb ¹⁸⁸Re-C1P5 mAb; B) CasKi tumor, control ¹⁸⁸Re-18B7 mAb; C) Hep 3B2.1-7 and A2058 human metastatic melanoma tumors, HBx-specific ¹⁸⁸Re-4H9 mAb.

FIG. 6A-6C. Radioimmunotherapy of CasKi tumors in nude mice: A) changes in tumor volume; B) mouse treated with 350 μCi ¹⁸⁸Re-C1P5 mAb; C) control mouse (both mice shown on Day 20 post-treatment).

FIG. 7A-7C. Radioimmunotherapy of Hep 3B2.1-7 tumors in nude mice: A) changes in tumor volume; B) control untreated mouse; C) mouse treated with 600 μCi ¹⁸⁸Re-4H9 mAb. Both mice shown on Day 18 post-treatment. In B and C lower panels show H&E stained tumors at the completion of the experiment.

### DETAILED DESCRIPTION OF THE INVENTION

The invention provides a method of treating a subject having a virus-associated cancer comprising administering to the subject a radiolabeled binding molecule, wherein the binding molecule binds to a virus antigen expressed by virus-associated cancer cells in the subject.

The invention also provides a method of imaging or treating virus-associated tumor cells in a subject comprising administering to the subject a radiolabeled binding molecule, wherein the binding molecule binds to a virus antigen expressed by virus-associated tumor cells in the subject.

The cancer cell can exhibits the virus antigen internally (intracellularly) and/or on the cell surface. Many cancer cells have virus antigens internally and/or on their surface or can be caused to have viral antigens internally and/or on their surface. As a result, a binding molecule that binds a virus antigen is preferentially bound within or to cells bearing the virus antigen. This binding permits specific targeting of cancer cells for treatment and/or imaging with binding molecules bearing radiation-emitting isotopes. Therefore, the virus antigen is preferably substantially absent from other cells of the subject. One having ordinary skill in the art will understand, however, that the virus antigen can and often is also present on pre-cancerous cells in a subject having cancer cells. Treatment of such pre-cancerous cells can eliminate virally infected cells before they transform into cancer cells.

Virus antigens are present on the surface of or within tumor cell by several pathways. Those having ordinary skill in the art will understand that several human neoplasias, carcinomas and dysplasias and many more animal cancers are caused by virus infections. For example, infection with human papilloma virus 16 (HPV16), Epstein-Barr virus (EBV), human T-cell lymphotrophic virus 1 (HTLV-1), bovine leukemia virus (BLV), feline leukemia virus (FeLV), Kaposi's sarcoma virus (KSV), Moloney murine sarcoma virus, Rous sarcoma virus (RSV), mouse mammary tumor virus, avian erythroblastosis virus, avian myeloblastosis virus, avian carcinoma virus, walleye dermal sarcoma virus (WDSV) and other oncogenic viruses are believed to directly contribute to or cause certain human or other mammalian cancers. In such cases, expression of virus-encoded genes, including virus-encoded glycoproteins and envelope proteins is a natural result of the infection. In preferred embodiments, the viral antigen is a virus-encoded membrane protein. The viral antigen is preferably a structural protein of a virus or a non-structural protein of a virus. The viral antigen is alternatively preferably the product of an oncogenic virus.

The viral antigen can also be the product of a non-oncogenic, optionally genetically modified virus. Therefore, the viral antigen is preferably the product of a natural viral gene or a non-natural viral gene. Non-oncogenic viruses that preferentially replicate in human cancer cells include the enveloped (−) RNA viruses vesicular stomatitis virus (VSV) in the family rhabdoviridae and Newcastle disease virus (NDV) in the family paramyxoviridae. NDV normally infects birds, whereas VSV naturally infects livestock and insects (Krishnamurthy et al. (2006) J Virol. 80:5145-5155; Zarate and Novella (2004) J. Virol. 78:12236-12242). Although currently under investigation as oncolytic agents, the ability of these viruses to preferentially replicate in human tumor cells also makes them suitable for targeted gene delivery (Fernandez et al. (2002) J. Virol. 76(2):895-904).

In some preferred embodiments, viral antigens become associated with tumor cells by targeted gene therapies based on these or other viral vectors using, e.g., unmodified, defective or engineered VSV, NDV, adenoviruses, herpesviruses, retroviruses, pseudo-typed viruses and others known in the art to deliver genes to tumor cells (see e.g., U.S. Pat. No. 7,090,837 to Spencer, et al. and references cited therein). Therefore, in some preferred embodiments, the viral antigen is the product of a natural gene of such a virus vector. In certain other preferred embodiments, the viral antigen is derived from a gene that has been inserted into a virus vector, and can be derived from a natural or non-natural gene carried by the virus vector.

A viral antigen-containing virus attached to a cellular virus receptor is also on the cell surface. In certain preferred embodiments, therefore, the viral antigen is bound to the surface of the cell by a virus receptor. Similarly, viral antigen-containing virus-like particles, virion fragments, virion subunits, or virus proteins bind to the cell surface as a result of interactions with the virus receptor or another cell surface binding site. Such a strategy would be useful where one prefers specifically labeling a tumor cell with non-infectious and/or purified virus fragments to direct the radiation-emitting isotope-bearing binding molecule to a tumor cell. Those having ordinary skill in the art will be familiar with methods of preparing and administering non-infectious preparations containing the viral antigen to a subject, so the virus antigen is alternatively part of a virus-like particle, virion fragment, virion subunit, or virus protein bound to the surface of a cell.

A bi-specific antibody can also bind both to a virus antigen-containing virus, virus-like particle or virus fragment and an epitope on a tumor cell, causing a virus to bind to a tumor cell when the virus might not otherwise. Such a strategy is particularly useful if the tumor cell lacks a cognate virus receptor. Preferably, a virus antigen is present on the surface of tumor cell as a result of interactions with a molecule that itself interacts with a tumor cell surface protein or surface oligosaccharide.

The cancer cell is preferably present in a solid tumor, a semi-solid tumor or a liquid tumor. The cancer cell is preferably a tumor cell. The cancer can be, for example, cervical carcinoma, hepatocellular carcinoma, a lymphoma, Burkitt's lymphoma, nasopharangeal carcinoma, Hodgkin's disease, skin cancer, primary effusion lymphoma, multicentric Castleman's disease, T-cell lymphoma, B-cell lymphoma, splenic lymphoma, adult T-cell leukemia, hary-cell leukemia, Kaposi's sarcoma, post-transplant lymphoma, brain tumor, multicentric Castleman disease, osteosarcoma, mesothelioma cervical dysplasia, anal cancer, cervical cancer, vulvar cancer, vaginal cancer, penile cancer, oropharyneal cancer, nasopharyneal cancer, oral cancer, liver cancer or skin cancer.

The virus antigen can be, for example, a product of hepatitis B virus (HBV), hepatitis C virus (HCV), Epstein-Barr virus (EBV), human herpes virus 8 (HHV8), a human papilloma virus (HPV), human papilloma virus 16 (HPV16), human papilloma virus 18 (HPV18), human papilloma virus 31 (HPV31), papilloma virus 33 (HPV33), papilloma virus 35 (HPV35), papilloma virus 39 (HPV39), papilloma virus 45 (HPV45), papilloma virus 51 (HPV51), papilloma virus 66 (HPV66), simian virus 40 (SV40), JC polyomavirus (JCV), BK polyomavirus (BKV), molluscum contagiosum virus (MCV), mouse mammary leukemia virus (MMLV), human adenoviruses A-F, cytomegalovirus (CMV), human T-cell lymphotrophic virus 1 (HTLV-1), human T-cell lymphotrophic virus 2 (HTLV-2), bovine leukemia virus (BLV), feline leukemia virus (FeLV), Kaposi's sarcoma virus (KSV), Moloney murine sarcoma virus , mouse mammary tumor virus, human immunodeficieny virus-1 (HIV-1), Rous sarcoma virus (RSV), vesicular stomatitis virus (VSV), Newcastle disease virus (NDV) , avian erythroblastosis virus, avian myeloblastosis virus, avian carcinoma virus or walleye dermal sarcoma virus (WDSV).

In a preferred embodiment, the cancer is cervical cancer and the virus antigen is a product of a human papilloma virus (HPV).

In certain other preferred embodiments, the virus is an enveloped virus. In certain preferred embodiments, the virus antigen is a product of a DNA virus, and the DNA virus is preferably a virus in the family hepadnaviridae, herpesviridae, papillomaviridae, polyomaviridae or poxviridae. The virus antigen is preferably a product of a virus selected from the group consisting of the hepatitis B virus (HBV), Epstein-Barr virus (EBV), human herpes virus 8 (HHV8), a human papilloma virus (HPV) such as HPV16, simian virus 40 (SV40) (Vilchez and Butel. (2004) Clin. Microb. Rev. 17:495-508; Butel (2000) Carcinogenesis 21:405-426), JC polyomavirus (JCV), BK polyomavirus (BKV), molluscum contagiosum virus (MCV), mouse mammary leukemia virus (MMLV), human adenoviruses A-F, cytomegalovirus (CMV), human T-cell lymphotrophic virus 1 (HTLV-1), bovine leukemia virus (BLV), feline leukemia virus (FeLV), Kaposi's sarcoma virus (KSV), Moloney murine sarcoma virus, and mouse mammary tumor virus.

In certain other embodiments, the virus antigen is a product of an RNA virus, and the RNA virus is preferably a member of the family rhabdoviridae, flaviviridae, paramyxoviridae, or retroviridae. The virus antigen is preferably a product of a virus selected from the group consisting of hepatitis C virus (HCV), HTLV-1, human immunodeficieny virus-1 (HIV-1), human papilloma virus 16 (HPV16), Epstein-Barr virus (EBV), human T-cell lymphotrophic virus 1 (HTLV-1), bovine leukemia virus (BLV), feline leukemia virus (FeLV), Kaposi's sarcoma virus (KSV), Moloney murine sarcoma virus, Rous sarcoma virus (RSV), mouse mammary tumor virus, VSV, and NDV.

In preferred embodiments, the binding molecule is a paratope-containing molecule, preferably an intact antibody, a monoclonal antibody, a polyclonal antibody, or a Fab antibody fragment. In certain embodiments, the binding molecule is a single chain polypeptide. In certain other embodiments, the binding molecule comprises the functional equivalent of a paratope derived from a non-immunoglobulin molecule that interacts with the viral antigen in other contexts. The functional equivalent of a paratope is preferably another virus component that interacts with the viral antigen in the virus life cycle, most preferably in the assembly and morphogenesis of a virion.

In different embodiments, the subject is preferably a mammal such as a mouse, a rat, a cat, a dog, a horse, a sheep, a cow, a steer, a bull, livestock, a primate, a monkey, and preferably a human, and can also be another animal such as a bird like a chicken or turkey, or a fish like a salmon or carp.

In preferred embodiments, the radiation-emitting isotope borne by the binding molecule is selected from the group consisting of 11-C, 18-F, 32-P, 34m-Cl, 38-K, 47-Sc, 51-Mn, 52-Mn, 52m-Mn, 52-Fe, 55-Co, 61-Cu, 62-Cu, 64-Cu, 67-Cu, 62-Ga, 67-Ga, 68-Ga, 72-As, 77-As, 75-Br, 76-Br, 82m-Rb, 83-Sr, 87-Sr, 86-Y, 89-Sr, 90-Y, 89-Zr, 94m-Tc, 99m-Tc, 105-Rh, 109-Pd, 111-Ag, 110-In, 111-In, 118-Sb, 120-1, 122-I, 123-I, 124-I, 125-I, 131-I, 177-Lu, 153-Sm, 159-Gd, 166-Ho, 166-Dy, 140-La, 194-Ir, 198-Au, 199-Au, 186-Re, 188-Re, 211-As, 212-Bi, 213-Bi, 212-Pb, 222-Ra, 223-Ra, 224-Ra, 225-Ra, 225-Ac, and 255-Fm.

The choice of the particular radioisotope with which the binding molecule is labeled can be determined by the size of the cancer to be treated and its localization in the body. Two characteristics are important in the choice of a radioisotope—emission range in the tissue and half-life.

Alpha emitters, which have a short emission range in comparison to beta emitters, can be preferable for treatment of small tumors that are disseminated in the body. Examples of alpha emitters include (half-life in parenthesis): 213-Bi (half-life 46 minutes), 223-Radium (half-life 11.3 days), 224-Radium (half-life 3.7 days), 225-Radium (half-life 14.8 days), 225-Actinium (half-life 10 days), 212-Lead (half-life 10.6 hours), 212-Bismuth (half-life 60 minutes), 211-Astatin (half-life 7.2 hours), and 255-Fermium (half-life 20 hours).

In a preferred embodiment, the alpha-emitting radioisotope is 213-Bismuth. 213-Bi emits a high linear energy transfer (LET) a-particle with E=5.9 MeV with a path length in tissue of 50-80 μm. Theoretically a cell can be killed with one or two a-particle hits. 213-Bi has been proposed for use in single-cell disorders and some solid cancers (McDevitt et al. (2000) Cancer Res. 60:6095-6100; Kennel and Mirzadeh (1998) Nucl. Med. Biol. 25:241-246; Behr et al. (1999) Cancer Res. 59:2635-2643; Adams et al. (2000) Cancer Biother. Radiopharm. 15:402a) and has been used to treat patients with leukemia in Phase I clinical trials (Kolbert et al. (2001) J. NucL Med. 42:27-32; Sgouros et al. (1999) J. Nucl. Med. 40:1935-1946). 213-Bi is the only a-emitter that is currently available in generator form, which permits transportation of this isotope from the source to clinical centers within the United States and abroad.

Beta emitters, with their longer emission range, can be preferable for the treatment of cancer cells in large tumors. Examples of beta emitters include 188-Rhenium (half-life 16.7 hours), 90-Yttrium (half-life 2.7 days), 32-Phosphorous (half-life 14.3 days), 47-Scandium (half-life 3.4 days), 67-Copper (half-life 62 hours), 64-Copper (half-life 13 hours), 77-Arsenic (half-life 38.8 hours), 89-Strontium (half-life 51 days), 105-Rhodium (half-life 35 hours), 109-Palladium (half-life 13 hours), 111-Silver (half-life 7.5 days), 131-Iodine (half-life 8 days), 177-Lutetium (half-life 6.7 days), 153-Samarium (half-life 46.7 hours), 159-Gadolinium (half-life 18.6 hours), 186-Rhenium (half-life 3.7 days), 166-Holmium (half-life 26.8 hours), 166-Dysprosium (half-life 81.6 hours), 140-Lantanum (half-life 40.3 hours), 194-Iridium (half-life 19 hours), 198-Gold (half-life 2.7 days), and 199-Gold (half-life 3.1 days). In a preferred embodiment, a beta-emitting radioisotope is 188-Rhenium. 188-Re is a long-range, high-energy β-emitter (Eₘₐₓ=2.12 MeV) that has recently emerged as an attractive therapeutic radionuclide in diverse therapeutic trials including cancer radioimmunotherapy, palliation of skeletal bone pain, and endovascular brachytherapy to prevent restenosis after angioplasty (Knapp (1988) Cancer Biother. Radiopharm. 13:337-349; Hoher et al. (2000) Circulation 101:2355-2360; Palmedo (2000) Eur. J. NucL Med. 27:123-130).

188-Re has the additional advantage that it emits gamma rays that can be used for imaging studies. For the treatment of cancer cells in large tumors or those in difficult to access sites deep in the body, longer-lived isotopes such as 90-Yttrium (half-life 2.7 days), 177-Lutetium (half-life 6.7 days) or 131-Iodine (half-life 8 days) can be preferred.

An advantage in using the same binding molecule processed in the same manner with multiple isotopes is another advantage that can be realized with the instant invention. 99m-Tc is a photon emitter for imaging that is a chemical analogue of 188-Re, giving the advantage that is can be used with the same binding molecules and attached by the same techniques, as by using the same chelating agent, as 188-Re. In a preferred embodiment of the invention, the radiation emitting isotope is 99m-Tc, 188-Re, or both.

Positron emitters can also be used, such as: 52m-Mn (21.1 min); 62-Cu (9.74 min); 68-Ga (68.1 min); 11-C (20 min); 82-Rb (1.27 min); 110-In (1.15 h); 118-Sb (3.5 min); 122-I (3.63 min); 18-F (1.83 h); 34m-C1 (32.2 min); 38-K (7.64 min); 51-Mn (46.2 min); 52-Mn (5.59 days); 52-Fe (8.28 h); 55-Co (17.5 h); 61-Cu (3.41 h); 64-Cu (12.7 h); 72-As (1.08 days); 75-Br (1.62 h); 76-Br (16.2 h); 82m-Rb (6.47 h); 83-Sr (1.35 days); 86-Y (14.7 h); 89-Zr (3.27 days); 94m-Tc (52.0 min); 120-1 (1.35h); 124-I (4.18 days). 64-Cu is a mixed positron, electron and Auger electron emitter.

Any of the radioisotopes, except alpha emitters, that are used for radioimmunotherapy can also be used at lower doses for radioimmunoimaging, for example a beta emitter, a positron emitter or an admixture of a beta emitter and a positron emitter. Preferred radioisotopes for use in radioimmunoimaging include 99m-Technetium, 111-Indium, 67-Gallium, 123-Iodine, 124-Iodine, 131-Iodine and 18-Fluorine. 111-Indium is a photon emitter for imaging use that is a chemical analogue of 213-Bi and can be coupled to binding molecules using the same techniques. For imaging, one can use an imaging-effective amount of a radiation-emitting isotope of about 1 to about 30 mCi for isotopes that are usually used diagnostically (e.g., 99m-Tc) and about 1 to about 5 mCi for isotopes that are usually used therapeutically (e.g. 213-Bi) to avoid unnecessary dosing to a patient.

The invention further provides a method for treating cancer cells in a subject that comprises the steps of administering to the subject an amount of binding molecules bearing a plurality of different radioisotopes effective to treat the cancer. In some embodiments, the radioisotopes are isotopes of a plurality of different elements. In a preferred embodiment, at least one radioisotope in the plurality of different radioisotopes is a long range emitter and at least one radioisotope is a short range emitter. Examples of long range emitters include gamma emitters, x-ray emitters, beta emitters and positron emitters. Examples of short range emitters include alpha emitters. Positron emitters can also be intermediate range emitters depending on the energy of the positrons. In a preferred embodiment, the long-range emitter is a beta emitter and the short range emitter is an alpha emitter. Preferably, the beta emitter is 188-Rhenium. Preferably, the alpha emitter is 213-Bi. Combinations of different radiation-emitting isotopes can be used, which include an admixture of any of an alpha emitter, a beta emitter, and a positron emitter, with physical half-lives from 2 minutes to 100 days. Preferably, the plurality of different radioisotopes is more effective in treating the tumor cell than a single radioisotope within the plurality of different radioisotopes, where the radiation dose of the single radioisotope is the same as the combined radiation dose of the plurality of different radioisotopes.

It is known from radioimmunotherapy studies of tumors that whole antibodies usually require from 1 to 3 days time in circulation to achieve maximum targeting. Although slow targeting does not impose a problem for radioisotopes with relatively long half-lives such as 188-Re (t_{1/2}=16.7 hours), faster delivery vehicles are needed for short-lived radioisotopes such as 213-Bi (t_{1/2}=46 min). Smaller binding molecules, including F(ab′)₂ and Fab′ fragments, provide much faster targeting that matches the half-lives of short-lived radionuclides (Milenic (2000) Semin. Radial. Oncol. 10:139-155; Buchsbaum (2000) Semin. Radiat. Oncol. 10:156-167). Use of short-lived binding molecules and radiation-emitting isotopes can further increase the safety of the method by reducing the period of time the subject is exposed to a potentially antigenic binding molecule and potentially radiotoxic isotope.

A therapeutically effective amount of a radiation-emitting isotope (dose) can vary depending on the localization and size of the tumor, the method of administration of the binding molecule bearing the radiation-emitting isotope (local or systemic) and the decay scheme of the isotope. In order to calculate the doses that can treat the cancer without radiotoxicity to vital organs, a diagnostic scan of the patient with the binding molecule radiolabeled with a diagnostic radioisotope or with a low activity therapeutic radioisotope can be performed prior to therapy, as is customary in nuclear medicine. The dosimetry calculations can be performed using the data from the diagnostic scan (Early and Sodee (1995) Principles and Practice of Nuclear Medicine, 2^{nd} ed. Mosby). For treatment of a subject having a virus-associated cancer, the radiolabled binding molecule is preferably administered in an amount that is effective to treat the cancer. In different embodiments, a therapeutically effective amount of a radiation-emitting isotope of the radioisotope for RIT is about 1 to about 1000 mCi.

RIT of cancer cells such as tumor cells has several advantages over the related immunotoxin approach where a binding molecule bears a toxin molecule, such as ricin or diphtheria toxin. With RIT, the binding molecule used for radiation delivery does not need to be internalized to kill the cell while toxins do. Also, with RIT, not every virus infected cell in the body needs to be targeted by the binding molecule. In vivo, there can be many tumor cells within a small three-dimensional space such that “cross-fire” radiation is effective. By contrast, toxins only kill the cell in which they enter. Consistent with this mechanism, 188-Re-labeled mAbs have been observed to be more effective in vivo than in vitro. In vitro, the “cross-fire” radiation is largely deposited on the two dimensional surface represented by the cell layer at the bottom of a tissue culture well.

In this regard, beta-emitting radionuclides that kill cells preferentially by “cross-fire” (Milenic et al. (2004) Nature Rev. Drug Discovery 3:488-498) have been successfully used for therapy of leukemia which is a single cell disease (Burke et al. (2002) Cancer Control 9: 106-113) and in pre-clinical RIT of systemic fungal infection (Dadachova et al. (2003) Proc. Natl. Acad. Sci. USA 100: 10942-10947; Dadachova et al. (2004) Antimicrob. Agents. Chemother. 48: 1004-1006; Dadachova et al. (2006) J. Infect. Dis. 193: 427-1436).

In contrast to toxin molecules, the radioisotope itself is unlikely to elicit significant immune responses that would limit subsequent use. RIT is also potentially less toxic since the chemistry of linking different radioisotopes to antibodies including ¹⁸⁸Re and ²¹³Bi has been well developed, and the exceptional stability of radiolabeled antibodies in vitro and in vivo has been confirmed.

As is known to those having ordinary skill in the art, availability of many isotopes differing in half-life and radiation type offers great versatility for designing RIT depending on the specific target and binding molecule (Milenic et al. (2004) Nature Rev. Drug Discovery 3: 488-498; Milenic et al. (2004) Cancer. Biother. Radiopharm. 19: 135-147; Dadachova et al. (2004) Proc. Natl. Acad. Sci. USA 101: 14865-14870). Nevertheless, combination therapy comprising RIT and another modality, such as chemotherapy, immunotoxins, or radiation-beam therapy may provide additional advantages than the RIT modality alone.

Based on the data accumulated in clinical RIT of cancer, the primary toxicity of RIT is likely to be bone marrow suppression. Important determinants of the extent and duration of myelosuppression include bone marrow reserve (based on prior cytotoxic therapy and extent of disease involvement), total infection burden and spleen size (Knox and Meredith (2000) Semin. Radiat. Oncol. 10:73-93; Hernandez and Knox (2003) Semin. Oncol. 30 (6 Suppl 17):6-100).

In addition, when using a radioactive therapy in patients there is always a concern of long-term effects such as the possible subsequent development of neoplasms arising from radiation-induced mutations. However, this risk should be extremely low after short-term exposure and should be outweighed by the benefits of treating the tumor.

It is also extremely unlikely that particulate radiation utilized in RIT will cause mutation of any virus whose genomic information is within a treated tumor cell. Viral replication already has an inherently high rate of mutation, and for RNA viruses, the mutation rate approaches the maximum rate compatible with continued viability (Drake (1993) Proc. Natl. Acad. Sci. USA 90:4171-4175). Increased mutation rates therefore can reduce viral fitness, especially for RNA viruses. The virus genome is also physically small compared to that of a tumor cell nucleus, and is less likely to be damaged directly by the emitted radiation. Furthermore, ionizing radiation is a weak mutagen in comparison with chemical mutagens abundant in the environment (Hall (2000) Radiobiology for the Radiologist, 5^{th} ed, Lippincott Williams & Wilkins).

Clinical data indicate that fractionated doses of radiolabeled antibodies and peptides are more effective than single doses against tumors and are less radiotoxic to normal organs (Dadachova et al. (2004) Antimicrob. Agents Chemother. 48:1004-1006; Lehrman (2005) Lancet 366: 549-555). Depending on the status of a patient and the effectiveness of the first treatment with RIT, the treatment can consist of one dose or several subsequent fractionated doses.

The radiolabeled binding molecule can be delivered to the subject by a variety of means used in medical or veterinary practice, including localized and systemic administration. Preferably, the radiolabeled binding molecule is administered parenterally. The radiolabeled binding molecule can be injected, for example, into the bloodstream, into a muscle or into an organ or body cavity.

The invention also provides a method of making a composition effective to treat a subject with cancer which comprises admixing a radiolabeled binding molecule and a carrier, wherein the binding molecule specifically binds to a virus antigen on and/or in virally-infected cancer cells. Such compositions can also be used to image the cancer in the subject.

As used herein, the term “carrier” encompasses any of the standard pharmaceutical carriers, such as a sterile isotonic saline, phosphate buffered saline solution, water, and emulsions, such as an oil/water or water/oil emulsions.

The invention further provides for the use of a radiolabeled binding molecule for the preparation of a composition for treating or imaging a subject with cancer, wherein the binding molecule is specific for a virus antigen that is on and/or in virally-infected cancer cells and wherein the radiolabeled binding molecule specifically binds to the virus antigen.

The invention also provides a method of using a radiolabeled binding molecule to image and/or treat virally-infected cancer cells which comprises:
(a) generating a binding molecule against a virus antigen expressed by a virally-infected cancer cell;
(b) attaching a radiolabel to the binding molecule; and
(c) administering to a subject an amount of the radiolabeled binding molecule effective to image and/or treat the virally-infected cancer cells.

The invention also provides a pharmaceutical composition formulated in dosage form, comprising a radiolabeled binding molecule and a pharmaceutically acceptable carrier, wherein the binding molecule is specific for a virus antigen expressed by a virally-infected cancer cell and wherein the dosage is appropriate to kill cancer cells that express the viral antigen in a subject or wherein the dosage is appropriate to image the cancer cells in a subject.

### Definitions

“Virus-associated cancer” refers to a cancer where viral infection is a co-factor in the cancer. Viruses linked to cancers in humans include the Epstein-Barr virus (EBV), which is associated with lymphomas and nasopharyngeal cancer; hepatitis B virus (HBV) and hepatitis C virus (HCV), which are both associated with cancer of the liver; human papillomaviruses (HPV), which are associated with cancer of the cervix; human T lymphotropic virus type 1 (HTLV-1) and type 2 (HTLV-2), which are associated with adult T-cell leukemia and with hairy-cell leukemia, respectively; and human herpesvirus 8 (HHV-8), which is associated with Kaposi sarcoma.

“Tumor cell” refers to a cell exhibiting abnormal cell growths, dysplasias, neoplasias and carcinomas in any form (i.e. in the form of a solid tumor, a benign tumor, carcinoma-in-situ, a dispersed carcinoma, a metastatic tumor cell, precancerous lesion, wart, or a dysplasia) in animals. In the context of the present invention, tumor cells include other cells having mis-regulated replication or development and specifically include cells comprising warts and pre-cancerous lesions.

An “antigen” is a molecule or part of a molecule comprising an epitope. An epitope is the binding site of a binding molecule, and includes those known in the art as conformational epitopes and linear epitopes. A “virus antigen” is an antigen encoded by a virus.

An antigen is on the cell surface if it is exposed to the extracellular environment or those intracellular spaces topologically equivalent to the extracellular space, such as the lumens of the ectoplasmic reticulum, golgi apparatus, and endocytic vesicles. An antigen is also on the cell surface with respect to this invention if it is outside the cell and is connected to the cell surface by another molecule.

As used herein, the term “treat” a subject with cancer means to kill cancer cells within the subject that express a virus antigen, to prevent the cancer from spreading in the subject, to reduce the further spread of cancer in the subject, to reduce the growth of a tumor in the subject, to prevent the growth of the tumor, to reduce the size of the tumor, and/or to eliminate the tumor or cancer in the subject. The term “treat” a tumor means to eradicate the tumor, to reduce the size of the tumor, to stabilize the tumor so that it does not increase in size, or to reduce the further growth of the tumor.

A “binding molecule” is a molecule capable of specifically binding an antigen. Binding molecules according to the present invention include paratopes, which are the antigen binding sites of the binding molecule, or the functional equivalent thereof. Exemplary and preferred binding molecules include antibodies and T-cell receptors. As used in the subject application, the term “antibody” encompasses whole antibodies and fragments of whole antibodies. Antibody fragments include, but are not limited to, F(ab′)₂ and Fab′ fragments, or generally, Fab fragments. F(ab′)₂ is an antigen binding fragment of an antibody molecule with deleted crystallizable fragment (Fc) region and preserved binding region. Fab′ is ½ of the F(ab′)₂ molecule possessing only ½ of the binding region. The term binding molecule is further meant to encompass polyclonal antibodies and monoclonal antibodies from any species as well as non-natural and engineered antibodies such as “humanized” murine antibodies, single-chain antibodies, and bi-specific antibodies such as those taught by U.S. Pat. No. 7,074,405 to Hansen, et al.

“Mab” or “mAb” means a single species of antibody according to this definition, most often a monoclonal antibody, but can include antibodies not produced by standard hybridoma techniques. Polyclonal antibody means a mixture including more than a single species of antibody. Preferred binding molecules include monoclonal antibodies and polyclonal antibodies as well as Fab fragments and engineered antibodies such as single chain antibodies, and humanized murine antibodies. Preferably, the binding molecule specifically binds to the viral antigen. Bi-specific antibodies can also be used. However, in one embodiment, the binding molecule is not a bi-specific antibody. Binding molecules need not be immunoglobulins or derived from immunoglobulins, however. Those having skill in the art will understand that there are functional equivalents of paratopes of immunoglobulins. Such equivalents are molecules or regions of molecules capable of binding a virus antigen on the surface of a tumor cell derived, for example, from other virus molecules that can bind the antigen.

The antibody used as a binding molecule can be any of an IgA, IgD, IgE, IgG, or IgM antibody. The IgA antibody can be an IgA1 or an IgA2 antibody. The IgG antibody can be an IgG1, IgG2, IgG2a, IgG2b, IgG3 or IgG4 antibody. A combination of any of these antibodies can also be used. One consideration in selecting the type of antibody to be used is the desired serum half-life of the antibody. IgG has a serum half-life of 23 days, IgA 6 days, IgM 5 days, IgD 3 days, and IgE 2 days (Abbas et al. Cellular and Molecular Immunology, 4^{th} edition, W.B. Saunders Co., Philadelphia, 2000). Another consideration is the size of the antibody. For example, the size of IgG is smaller than that of IgM allowing for greater penetration of IgG into tumors. IgA, IgG, and IgM are preferred antibodies.

Many binding molecules useful for the present methods have been described. Preferred antibodies include antibodies mAb C1P5 to HPV16 and HPV18 E6 (Abcam, Cambridge, Mass., USA), mAbs PRH-7A, PRH-3, PRH-4 to HTLV-1 gp46, mAb 72A1 against EBV envelope gp350, mAb 13B10 against human herpesvirus 8 (HHV-8) latent nuclear antigen-1 (LNA-1), and MAb D4.12.9 against the HCV E2 protein. Other preferred antibodies are specific for EBV Ea-R protein p17, a mouse IgG₁ specific for HHV-8 K8.1 gene products (K8.1A and K8.1B envelope proteins), and a mouse IgG₂ₐ specific for ORF K8.1A (envelope) from Advanced Biotechnologies Inc. (Columbia, Md.). Still further preferred binding molecules are provided in the Examples below.

It is to be understood that a binding molecule useful herein need not be and preferably is not a so-called “neutralizing” antibody or binding portion thereof. Thus, it is preferred that the binding molecule not itself kill or be responsible for killing a cancer cell that exhibits a viral antigen. Mere specific binding to that antigen is sufficient for the purposes contemplated here.

The binding molecule can be radiolabeled with a radiation-emitting isotope using, e.g., one of two techniques—“direct” radiolabeling or radiolabeling through a bifunctional chelating agent. As described previously (Dadachova et al., U.S. Patent Publication Nos. 2004/0115203 A1 and 2006/0039858 A1), 225-Actiunium (225-Ac) for construction of a 225-Ac/213-Bi generator can be acquired from Oak Ridge National Laboratory (Oak Ridge, Tenn.). The 225-AC/213-Bi generator is constructed using a MP-50 cation exchange resin, and 213-Bi is eluted with 0.15 M HI (hydroiodic acid) in the form of 213-BiI₃ as described by Boll et al. (Radiochim. Acta 79:145-149, 1997). ¹¹¹InCl₃ can be purchased from Iso-Tex Diagnostics (Friendswood, Tex.). Binding molecules can be radiolabeled with, for example, 213-Bi (for therapy) or 111-In (for imaging) via the bifunctional chelator CHXA″ (N[2-amino-3-(p-isothiocyanato-phenyl)propyl]-trans-cyclohexane-1,2-diamine-N,N′,N″,N′″,N″″-pentaacetic acid) (Saha, Fundamentals of Nuclear Pharmacy, (1997) Springer, N.Y., pp.139-143). The average number of chelates per antibody can be determined by the Yttrium-Arsenazo III spectrophotometric method (Dadachova et al. (1997) Appl. Rad. Isotop. 48:477-481; Pippin et al. (1992) Bioconjug. Chem. 3:342-345). If required, the radiolabeled antibodies can purified by size exclusion HPLC (TSK-Gel.RTM. G3000SW, TosoHaas, Japan). Binding molecules can also be labeled, e.g., with 188-Re and 99m-Tc via “direct labeling” through reduction of antibody disulfide bonds with dithiothreitol (Dadachova and Mirzadeh, Nucl. Med. Biol. (1997) 24:605-608). 188-Re in the form of Na perrhenate (Na 188-ReO₄) can be eluted from a 188-W/188-Re generator (Oak Ridge National Laboratory, Oak Ridge, Tenn.). MAbs can be labeled with 188-Re via reduction of antibody disulfide bonds by incubating the antibody with 75-fold molar excess of dithiothreitol for 40 minutes at 37° C. followed by centrifugal purification on Centricon-30 or -50 microconcentrators with 0.15 M NH₄OAc, pH 6.5. Simultaneously with 3-10 mCi (110-370 MBq) 188-ReO₄ in saline can be reduced with SnCl₂ by incubation in the presence of Na gluconate, combined with purified reduced antibodies and kept at 37° C. for 60 minutes. Radioactivity not bound to the antibody can be removed by centrifugal purification on Centricon microconcentrators (Millipore Corp. Billerica, Mass.). Na^{99m}TcO₄ can be purchased from GE Healthcare (Bronx, N.Y.) and attached to binding molecules as described for 188-Re.

Although this invention has been described with an emphasis upon preferred embodiments, it will be obvious to those of ordinary skill in the art that variations of the preferred compounds and methods can be used and that it is intended that the invention can be practiced otherwise than as specifically described herein. Accordingly, this invention includes all modifications encompassed within the spirit and scope of the invention as defined by the following claims.

“A,” “an” and other singular terms are intended to include the plural forms thereof unless specifically excluded, and plural forms are intended to include the singular unless specifically excluded.

### EXPERIMENTAL DETAILS

It has been estimated that nearly 20% of human cancers worldwide have an infectious etiology (Parkin DM (2006) Int J Cancer 118:3030-3044.). Most of these tumors are of viral origin, and include firmly established associations of hepatitis B virus (HBV) and hepatitis C virus (HCV) with hepatocellular carcinoma; and of human papillomavirus (HPV)—with cancers of the cervix, anus, vulva, vagina; as well as associations of oropharynx Epstein-Barr virus (EBV) with lymphoma and nasopharyngeal carcinoma; human T lymphotropic virus type 1 (HTLV-1)—with adult T-cell leukemia/lymphoma, and human herpes virus 8 (HHV-8)—with Kaposi sarcoma (Cesarman E and Mesri EA (2007) Curr Top Microbiol Immunol 312:263-287; Jones EE and Wells SI (2006) Curr Mol Med 6:795-808; El-Aneed A and Banoub J (2006) Anticancer Res 26:3293-3300; Arbach H et al. (2006) J Virol 80:845-853; Young LS and Murray PG (2003) Oncogene 22:5108-5121; Mortreux F et al. (2003) Leukemia 17:26-38). In combination, these virus-associated tumors represent a burden of approximately 1.3 million cases of cancer each year, with HBV/HCV-associated liver cancer accounting for 523,000 cases, and HPV-associated tumors accounting for 561,000 cases (Parkin et al. (2005) CA Cancer J Clin 55: 74-108). The need to find new approaches to the treatment and prevention of virus-associated cancers is obvious and urgent.

### Radioimmunotherapy of Virus-Associated Cervical Cancer and Hepatocellular Carcinoma

The invention provides a novel strategy against tumors caused by viruses that uses binding molecules such as mAbs to viral proteins to deliver tumoricidal radiation. This strategy is fundamentally different from prior uses of radioimmunotherapy (RIT) which have targeted tumor associated antigens that are “self” proteins.

Human papillomavirus (HPV) causes cervical cancer, which according to the World Health Organization (WHO) (2006) is the second biggest cause of female cancer mortality worldwide. WHO estimates the number of cervical cancer deaths to be around 250,000 per year. Genotypes of HPV can be grouped into “high-risk” and “low-risk” types according to the degree of risk of development of cancer after infection with each genotype. A subset of women with high-risk HPVs such as HPV-16 or HPV-18 will develop preneoplastic lesions of cervical intraepithelial neoplasia. The minority of lesions that progress to high-grade dysplasias tend to persist and/or progress to carcinomas in situ before becoming invasive cancers. According to the WHO, the majority of adenocarcinomas of the cervix and of squamous cell cancers (SCC) of the vulva, vagina, penis and anus are caused by HPV-16 and HPV-18 (together accounting for about 70% of cases globally), the remaining 30% being due to other high-risk HPV types (such as HPV-31, -33, -35, -39, -45,-51, -66). The relative importance of different high-risk types varies between countries and regions, but type 16 has the greatest contribution to cervical cancer in all regions. HPV is also associated with other cancers of the anus, head and neck, and rarely, recurrent respiratory papillomatosis in children (WHO, 2006).

In the USA, cervical cancer has a major impact on women's health with 10,500 new cases being diagnosed in the USA in 2004 (American Cancer Society statistics). Human papilloma viruses (HPVs) HPV16 and HPV18 are associated with approximately 90% of cervical cancers. Mutational analysis has shown that the E6 and E7 genes of the high risks HPVs are necessary and sufficient for HPV transforming function. Thus, targeting the E6 and E7 proteins on transformed cells with cytocidal agents serves as a strategy for treatment and/or prevention of cervical cancer.

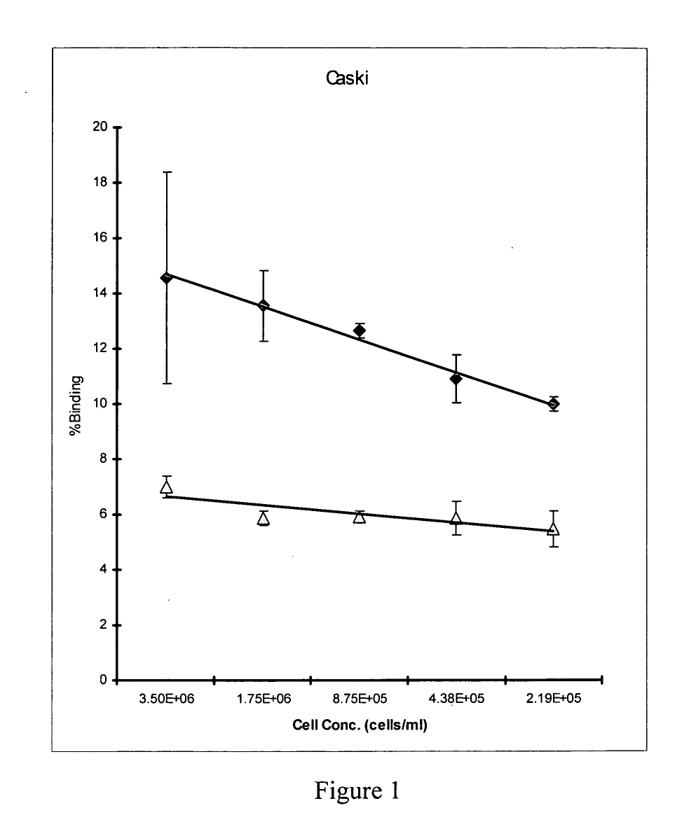

Experiments were conducted to determine the suitability of targeting HPV 16 and HPV18 using RIT with the commercially available C1P5 mAb to E6 (IgG1 isotype, Abcam, Cambridge Mass.) radiolabeled with 188-Rhenium (¹⁸⁸Re) and the HPV-16 expressing CasKi human cervical carcinoma cell line. The binding of ¹⁸⁸Re-C1P5 mAb to whole CasKi cells was compared to binding of ¹⁸⁸Re-labeled isotype-matching control mAb 18B7. There was 16% binding of ¹⁸⁸Re-C1P5 to whole CasKi cells as compared to 6% for ¹⁸⁸Re-18B7 (FIG. 1). Not every cell in a tumor needs to targeted for binding for effective therapy as cells adjacent to the targeted cells or even distant cells (depending on the emission range of the radionuclide) will be killed through the so called “cross fire” effect.

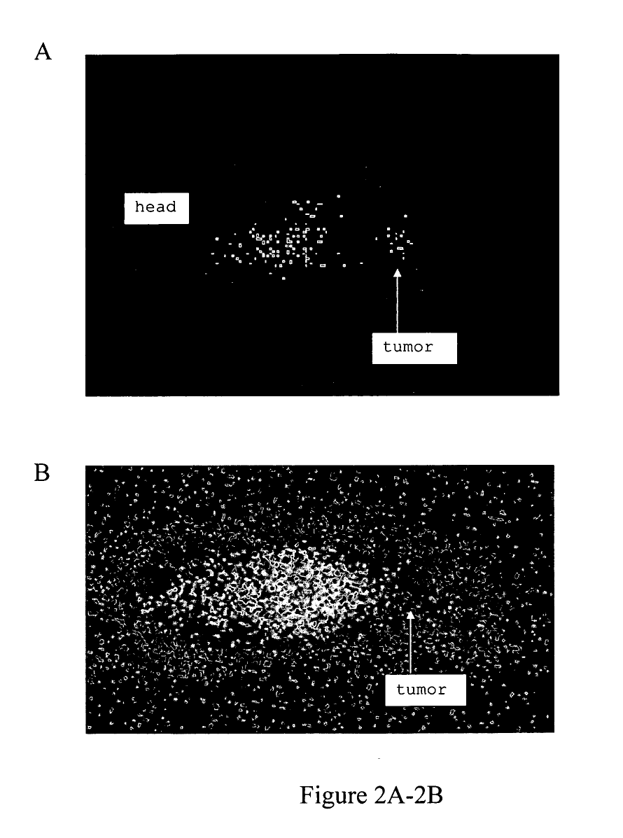

For evaluating the in vivo uptake of ¹⁸⁸Re-C1P5 mAb, CasKi tumors were induced in nude mice by injecting 10⁷ cells subcutaneously. When the tumors reached 4-5 mm in diameter, mice were injected IP with ¹⁸⁸Re-C1P5 mAb or with ¹⁸⁸Re-18B7 control mAb. Scintigraphic imaging of the animals was performed at 24 hr post-injection and biodistribution studies were conducted at 48 hr. There was a visible uptake in the tumors with ¹⁸⁸Re-C1P5 on the scintigraphic images, and no visible uptake with control mAb ¹⁸⁸Re-18B7 (FIG. 2A-B). Calculation of the tumor to normal muscle ratio from the biodistribution results showed that the difference between ¹⁸⁸Re-C1P5 and ¹⁸⁸Re-18B7 was impressive—10:1 versus 3:1, respectively. The overall uptake of ¹⁸⁸Re-C1P5 mAb in the tumors at 48 hr post-injection was 2.0 (±0.3)% of the injected dose per gr. The level of uptake of ¹⁸⁸Re-C1P5 may be caused by low numbers on non-viable and/or apoptotic cells in the tumors in which E6 antigen becomes accessible to ¹⁸⁸Re-C1P5 mAb. Ways to counteract low levels of accessible antigen include pre-treating the tumor cells either with external ionizing radiation, which increases the number of apoptotic and necrotic cells, and/or with a proteasome inhibitor MG132, which causes an increase in the levels of E6 and E7 proteins (Oh, KJ et al. J Virol. 78, 5338 (2004); Kehmeier, E et al. Virology 299, 72 (2002)). Alternatively, a mAb with a higher specificity than C1P5 could be used.

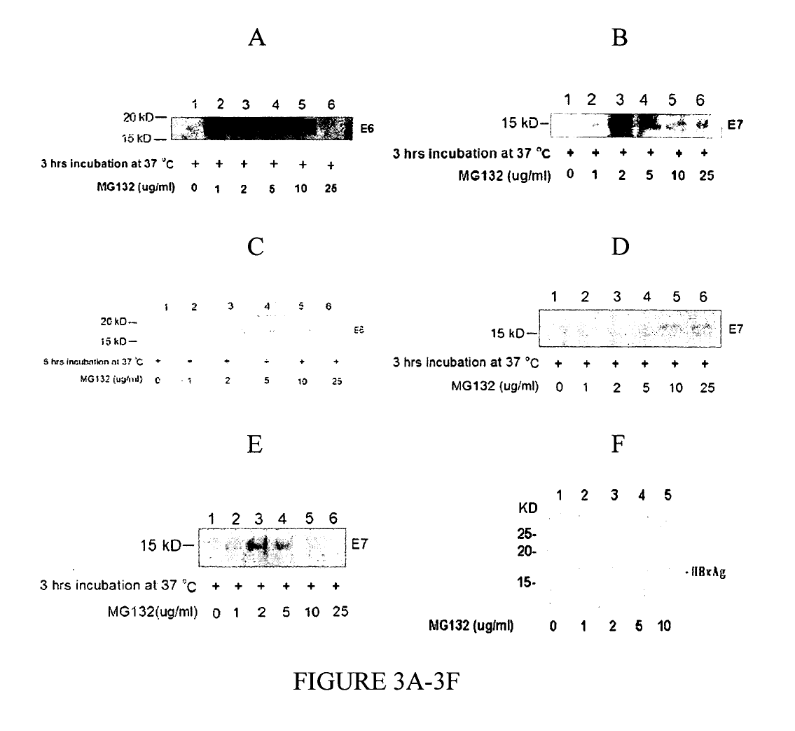

The expression of E6 and E7 was assessed by Western blot in three human cervical carcinoma cell lines—HPV16-positive CasKi and SiHa cell lines and HPV18-positive HeLa S3 cell line. While CasKi cells expressed both E6 and E7 antigens (FIG. 3A,B), SiHa and HeLa S3 cell lines had no measurable expression of E6 antigen (results not. shown) but did express E7 protein (FIG. 3D, E); albeit, the level of E7 expression was low in SiHa cells (FIG. 3D).

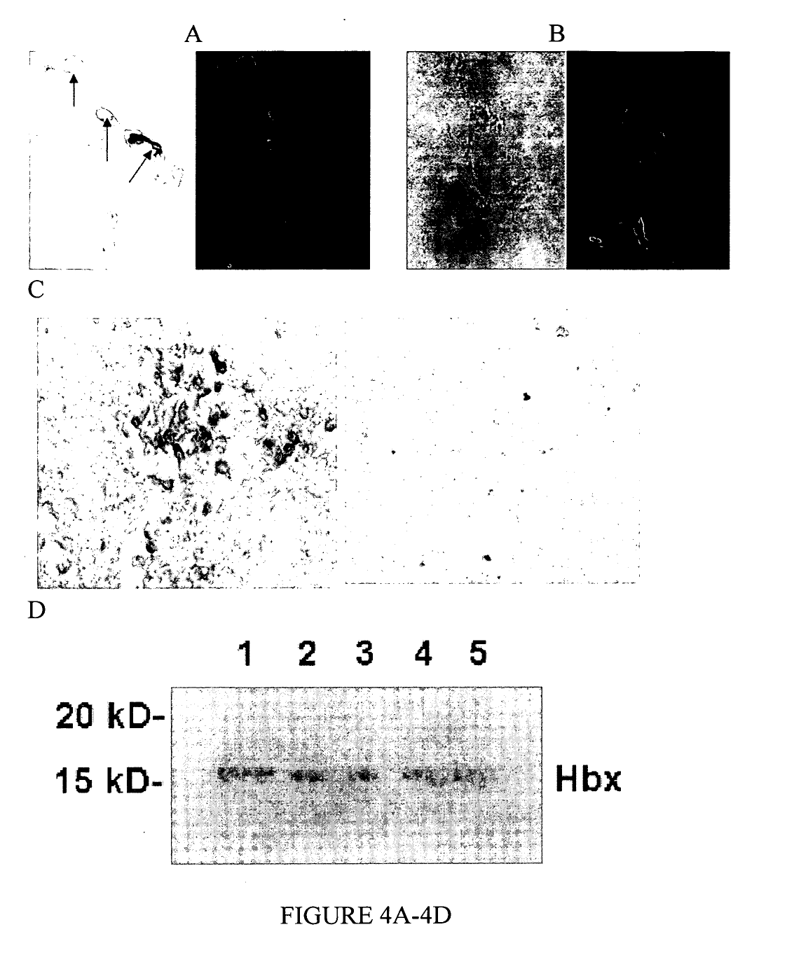

The proteasome inhibitor MG132 reduces the degradation of ubiquitin-conjugated proteins in mammalian cells without affecting ATPase or isopeptidase activities. MG132 has been reported to result in increased levels of E6 and E7 proteins in cervical cancer cells (Kehmeier et al. (2002) Virology 299:72-87; Oh et al. (2004) J Virol 78: 5338-5346). As higher amounts of target antigens can potentially improve RIT results, the influence was investigated of pre-treatment of cervical cancer (CC) cells with proteasome inhibitor MG132 on the levels of E6 and E7 expression. FIGS. 3A and B shows that in CasKi cells pre-treatment with MG132 caused an increase in expression of both E6 and E7, with the greatest level of expression achieved with use of 2 and 5 μg/mL of MG132, which subsided when higher doses were used. Prolongation of the incubation period of CasKi cells with MG132 did not result in greater E6 expression. In fact, decreased E6 expression was observed with prolonged CasKi cell exposure to MG132 (FIG. 3C), which might reflect an increase of protein degradation after more than 3 hr of incubation. Similar experiments were performed for E7 protein in SiHa and HeLa S3 cell lines (FIG. 3D, E). SiHa cells did not demonstrate an appreciable increase in the E7 levels (FIG. 4D); whereas, E7 levels did increase slightly for HeLa S3 cells treated with 1 and 2 μg/mL MG132 (FIG. 3E). Given reliable and high expression of E6 in CasKi cells as well as their ability to produce tumors in nude mice—CasKi cells and E6 protein were selected for further in vitro and in vivo experiments.

### Selection of a Cell Line—Antigen Combination to Act as an Experimental Hepatitis B-Associated Hepatocellular Carcinoma (HCC) Model

Two Hepatitis B-associated viral proteins were evaluated as potential targets for RIT—HBx and PreS2. HBx is suspected to have a role in hepatocarcinogenesis and, unlike other potential choices, HBx has no homology to host proteins. Further, the gene that codes for HBx is retained even when the HBV genome becomes integrated in hepatocellular carcinoma (HCC), whereas other HBV genes may be lost (Hwang et al. (2003) J Clinical Microbiol 41:5598-5603; Lupberger J and Hildt E (2007) World J Gastroenterol 13:74-81). PreS2 is also suspected to have a role in hepatocarcinogenesis (i.e., through transactivation of cellular genes important in growth control) (Lupberger J and Hildt E (2007) World J Gastroenterol 13:74-81). HBx protein was consistently detected by Western blot of HCC cell line Hep 382.1-7 using 4H9 mAb, and its expression was independent of pre-treatment with MG132 proteasome inhibitor (FIG. 3F), whereas preS2 was not detected (results not shown). Therefore, the Hep 3B2.1-7 cell line and HBx protein combination were selected for further experiments.

### Binding of Antibodies to Viral Antigens in Non-Viable Cells

To find out if antibodies to viral proteins which were identified as targets for RIT will be able to bind to viral proteins in non-viable tumor cells, immunofluorescence was performed of fixed and permeabilized CasKi and Hep 3B2.1-7 cells with mAbs C1P5 to E6 and 4H9 to HBx proteins, respectively, followed by FITC-conjugated polyclonal antibody to mouse IgG. While the binding of C1P5 mAb to the cells that were almost intact was weak, the heavily damaged cells with penetrable membranes showed bright fluorescence pointing to binding of C1P5 to E6 (FIG. 4A). The fixation and permeabilization also made possible for mAb 4H9 to bind to HBx protein (FIG. 4B). No binding of control IgG1 mAb to fixed and permeabilized CasKi or Hep 3B2. 1-7 cells was observed (results not shown).

### Expression of Viral Proteins in the Tumors

To confirm that CasKi and Hep 3B2.1-7 cells continued to express E6 and HBx viral antigens, respectively, in the tumors induced in nude mice, immunohistochemistry and immunoblot were performed for E6 and HBx, respectively. Western blot was chosen for Hep 3B2.1-7-induced tumors based on the literature reporting difficulties in reliably detecting HBx protein in organs by immunohistochemistry (Reifenberg K et al. (1999) J. Virol. 73:10399-10405). There was intense staining of E6 with specific mAb C1P5 in CasKi tumors (FIG. 4C, left panel) with no staining observed with control IgG1 mAb (FIG. 4C, right panel). Western blot of Hep 3B2.1-7-induced tumors revealed the presence of HBx protein (FIG. 4D). Thus, the presence of the target viral proteins in CC and HCC experimental tumors provided the possibility of targeting these antigens in vivo with radiolabeled mAbs for scintigraphic imaging and therapy.

### Biodistribution of Radiolabeled MAbs to Viral Proteins in CasKi and Hep 3B2.1-7-Tumor Bearing Nude Mic

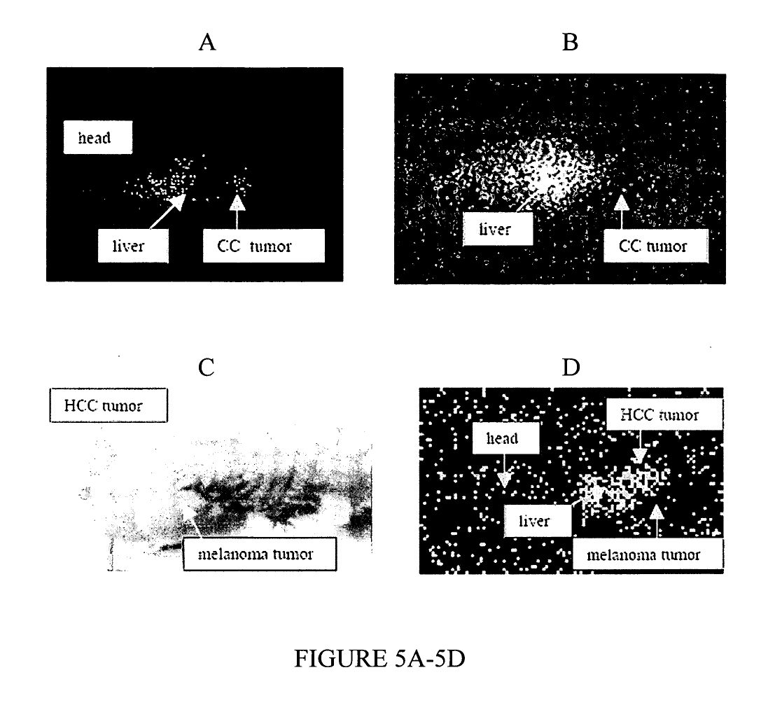

Imaging and biodistribution experiments were performed with ¹⁸⁸Re-radiolabeled C1P5 and 4H9 mAbs in CasKi and Hep 3B2.1-7-tumor bearing nude mice, respectively, to ascertain the localization of mAbs to the tumors. At 24 hr post-injection the CasKi tumor was visible on the scintigraphic image of a mouse injected with ¹⁸⁸Re-C1P5 mAb (FIG. 5A) as opposed to the image of a mouse injected with irrelevant ¹⁸⁸Re-18B7 mAb (FIG. 5B). The tumor to muscle ratio was calculated from the 48 hr biodistribution results which was 10:1 for ¹⁸⁸Re-C1P5 versus 3:1 for ¹⁸⁸Re-18B7. The overall uptake of ¹⁸⁸Re-C1P5 mAb in the tumors at 48 hr post-injection was 2.0(±0.3)% of the injected dose per gram tumor. Other organs like liver, spleen and blood showed the levels of uptake characteristic of IgG1 mAbs. For Hep 3B2.1-7 another approach was utilized to prove the specificity of mAb uptake in the tumor by using a model when a mouse carries two different tumors—one the tumor of interest and another—irrelevant control tumor. Nude mice carried A2058 human metastatic melanoma tumor on the right flank and Hep 3B2.1-7 on the left (FIG. 5C). The mice were injected with ¹⁸⁸Re-4H9 mAb and imaged scintigraphically at 24 hr post-injection. The antibody localized to the Hep 3B2.1-7 tumor as opposed to the irrelevant control melanoma tumor (FIG. 5D). The ability of radiolabeled mAbs to viral antigens to localize to these tumors in mice justified an assessment of RIT in these in vivo cancer models.

### RIT of CasKi and Hep 3B2.1-7-Tumor Bearing Nude Mice

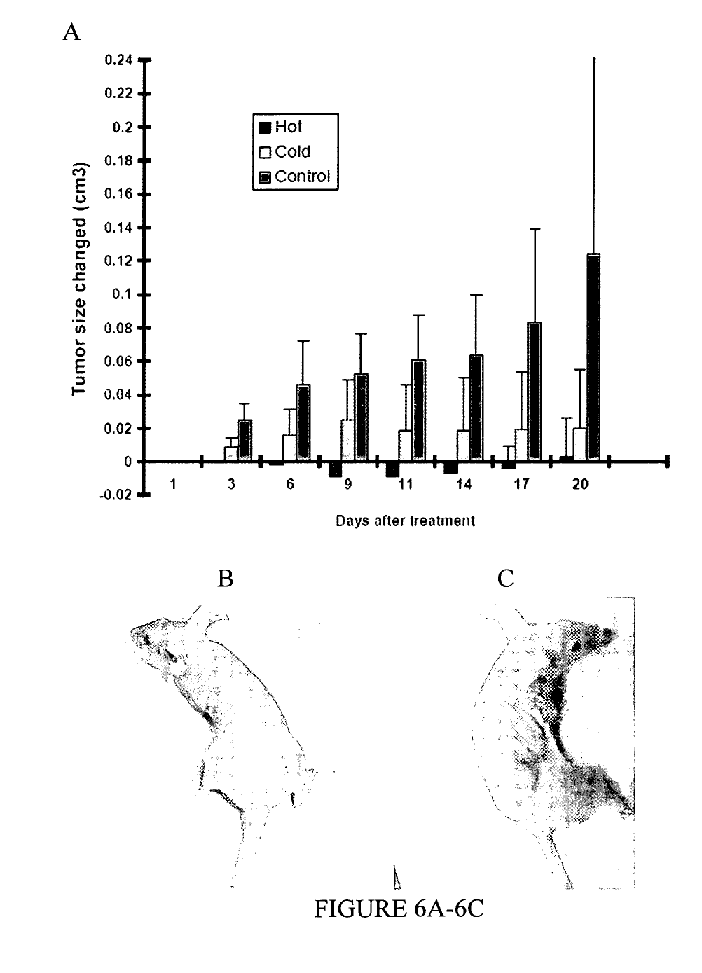

To evaluate the therapeutic effect of ¹⁸⁸Re-C1P5 mAb in CasKi tumor-bearing mice, the animals were injected with either 350 μCi ¹⁸⁸Re-C1P5 mAb, or matching amounts (30 μg per mouse) of unlabeled (“cold”) C1P5 mAb or left untreated. FIG. 6A shows the change in tumor volume in the treated and control groups. RIT with 350 μCi ¹⁸⁸Re-C1P5 mAb completely arrested tumor growth and resulted in its volume reduction (FIG. 6A and B), while untreated tumors grew aggressively (FIG. 6A and C) and untreated control mice had to be sacrificed on Day 20 post-treatment (P<<0.01). Interestingly, administration of “cold” C1P5 mAb also resulted in significant retardation of the tumor growth, which can be due to the induction of inflammation and complement cascades by the mAb.

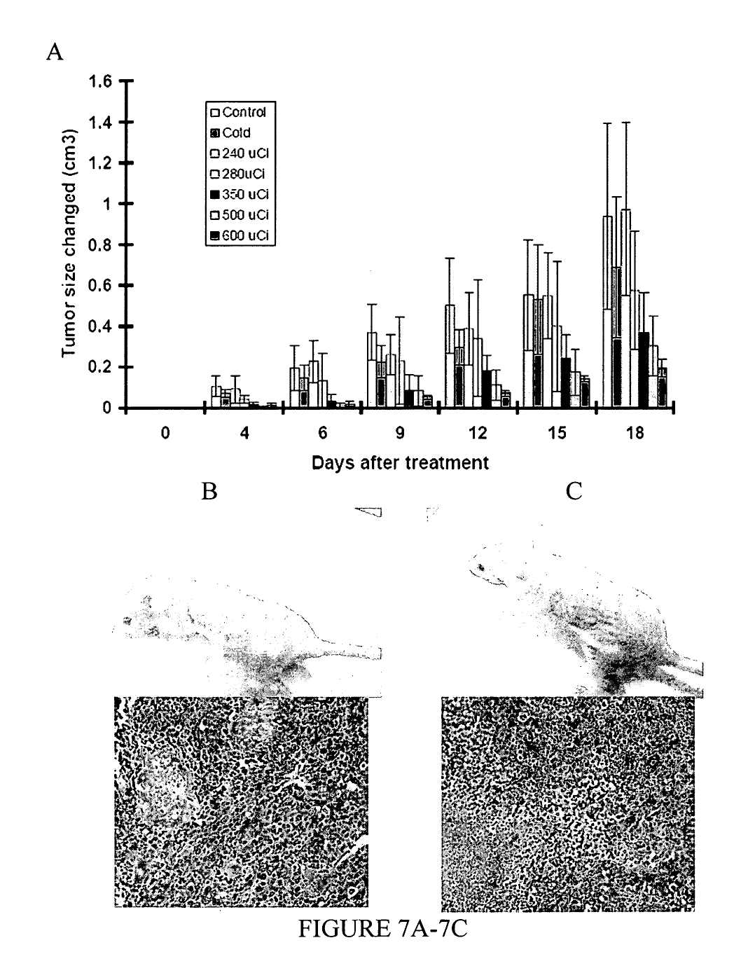

For RIT of Hep 3B2.1-7 tumors in nude mice, the same experimental design was initially employed as for CasKi tumors by using 350 μCi ¹⁸⁸Re-4H9 mAb, “cold” mAb-treated controls and untreated groups. Administration of 350 μCi ¹⁸⁸Re-4H9 mAb resulted in slower tumor growth but did not arrest it like in case of CasKi tumors. “Cold” 4H9 mAb did not have an effect on the tumor growth. To find the most efficient dose of radiolabeled mAb for retarding the growth of very aggressive Hep 3B2.1-7, a dose response experiment was done. The therapeutic effect of ¹⁸⁸Re-4H9 mAb began to manifest itself at a dose of 280 μCi and the effect increased with every subsequent increase in the dose (FIG. 7A) (P<0.02). At the completion of the experiment, the tumors from the control untreated mice and mice in the highest 600 μCi group were analyzed histologically. The control tumor consisted of moderately differentiated hepatoid cells with scattered small foci of necrosis, fibrin thrombosis, and hemorrhage. Neoplastic cells had ample eosinophilic to finely vacuolated cytoplasm and medium-sized nuclei to very large and anaplastic nuclei containing multiple large eosinophilic nucleoli with multinucleated cells occasionally evident. The mitotic index was high and there were scattered apoptotic cells (FIG. 7B). In contrast, the RIT-treated tumors had significantly more necrosis and hemorrhage than seen in the controls. The morphologic appearance of the tumor cells often had a more vacuolated cytoplasm suggesting degeneration (FIG. 7C).

### Discussion

Proof-of-principle in vitro and in vivo experiments described herein established the feasibility of targeting viral antigens in tumors of viral etiology with radiolabeled antibodies for therapy. Experimental cervical carcinoma (CC) and hepatocellular carcinoma (HCC) models were used, since these cancers are etiologically related to HPV and HBV/HCV, respectively, and have major public health implications world-wide.

The first challenge was the choice of target viral antigens in CC and HCC. In HPV-associated CC E6 and E7 oncoproteins were identified as potential targets for MT, because they are thought to be expressed in all cervical cancer cells; whereas, other viral genes may be lost during the multi-stage process of tumorigenesis. Similarly, in HCC, HBx expression is thought to be maintained. There is, however, an important concern in targeting these three proteins with radiolabeled mAbs—their subcellular location. Both E6 and E7 are located within the nucleus, and HBx is found in the nucleus and occasionally in the cytoplasm. As a consequence, the radiolabeled mAbs to these proteins can bind only to their respective antigens when they are released from the dead cells or when tumor cells are permeabilized. The CasKi cell line was chosen as a CC model, since it expressed E6 and E7 at high levels in vitro and the tumors grew aggressively in nude mice after the latent period. The Hep 3B2.1-7 cell line was chosen as a HCC model, since it expressed HBx at high levels and displayed fast tumor growth in mice.

The immunofluorescence of the tumor cells and immunohistochemistry and Western blot of the tumors revealed ample amounts of E6 and HBx antigenic targets in CasKi- and Hep 3B2.1-7-induced tumors, respectively (FIG. 4). The presence of accessible antigen for the radiolabeled mAbs is probably a result of protein release from tumor cells undergoing rapid turnover. Consequently, the radiolabeled antibodies accumulated in the tumor tissue such that they could be imaged scintigraphically (FIG. 5). One of the advantages of targeting viral antigens in the tumors that they are only found in malignant cells. In contrast, Chen and colleagues (Cancer Res 49: 4578-4585, 1980) observed no differences in the tumor uptake for both specific and non-specific radiolabeled antibodies in their biodistribution experiments when they targeted intranuclear histones

### The Ability of Radiolabeled MAbs Against Viral Proteins to Deliver Cytotoxic Radionuclide

188-Rhenium to the tumor cells facilitated the successful outcomes of RIT with these mAbs in tumor-bearing mice. The administration of 350 μCi dose of E6-binding ¹⁸⁸Re-C1P5 mAb to CasKi tumor-bearing mice completely stopped tumor growth and even resulted in its regression (FIG. 6). It is also likely that there was some added therapeutic benefit from, the antibody itself as unlabeled antibodies can mediate inflammatory reactions and activate compliment. The dose response experiments in mice bearing Hep 3B2.1-7 HCC-tumors clearly demonstrated a dose dependence for the therapeutic effect (FIG. 7). The fact that higher doses of radiolabeled mAb were needed to produce a therapeutic effect in HCC than in cervical tumor might reflect the aggressiveness of Hep 3B2.1-7 and the known radioresistance of liver tumors in comparison to the relatively radiosensitive cervical carcinomas (Yang et al. (2005) World J Gastroenterol 11: 4098-4101). It is also noteworthy that therapeutic gains in both experimental tumors were achieved with doses of radioactivity that are well below the maximum tolerated dose of approximately 800 μCi for ¹⁸⁸Re-labeled IgG1s in mice (Sharkey et al. (1997) Int J Cancer 72:477-485), and as such the doses used were not expected to produce any short- or long-term toxicities.

It is important to emphasize that when treating viral-associated cancers by targeting viral antigen not every cell in the tumor needs to express viral antigens for a therapeutic effect. Long range emitters such as ¹⁸⁸Re (emission range in tissue 10 mm) emit radiation in a 360° sphere and consequently can kill cells in the vicinity of the antigen location. Furthermore, a high concentration of the targeted viral antigen is probably not required for the delivery of a therapeutic dose to the tumor, according to a recent model of RIT for melanoma (targeted against melanin which is also an intracellular antigen). This model showed that the radiation dose delivered to the tumor is largely independent of melanin (antigen) concentration (Schweitzer et al. (2007) Melanoma Res 17:291-303).

This approach also holds the promise to prevent viral-associated cancers in chronically infected individuals—for example, persistence of HPV infection in HIV-infected individuals puts them at significant risk of developing HPV-associated cancers. While there is immunotherapy for HBV and HCV, many patients do not achieve viral clearance and the treatment is associated with substantial morbidity. There is also no vaccine for HCV, and many millions of people worldwide are infected with HBV despite the availability of an effective vaccine. Cells persistently infected with HPV, HBV, HCV or other viruses could potentially be eliminated with RIT targeted to viral antigens before they transform into malignant phenotype.

In conclusion, in vitro and in vivo experiments exemplified a novel strategy to treat virus associated tumors using radiolabeled mAbs targeted against viral proteins. This strategy is fundamentally different from prior uses of RIT in oncology that target tumor-associated human antigens thus resulting in significant uptake of radioactive antibody in normal tissues, leading to toxicity. The results of the present study indicate that by targeting instead viral and not self-proteins, radiolabeled mAbs may concentrate more specifically within tumor tissue, resulting in greater efficacy and less toxicity. This strategy also raises an exciting additional possibility to prevent virus-associated cancers in chronically infected patients by eliminating cells infected with oncogenic viruses before they transform into cancer.

### Materials and Methods

Antibodies. Mouse mAbs from Abcam C1P5 to HPV16 E6+HPV18 E6 and TVG 701Y to HPV16 E7 were used in CC experiments. Mouse mAb 4H9 to HBV HBx (Aviva, Cat# AVAMM2005) and mouse mAb S26 to HBV protein Hbs (Gene Tex Inc. Cat# GTX18797) which cross-reacts with HBsAg preS2 antigen were used in HCC experiments. Murine 18B7 mAb (IgG1) to C. neoformans (Casadevall A et al. (1992) J Infec Dis 165:1086-1093) was used as an irrelevant control for all specific antibodies. Rabbit polyclonal antibody conjugated with alkaline phosphatase to mouse IgG H&L was purchased from Abcam and used as secondary antibody in Western blot.

Cell lines and cell cultivation. Three human cervical carcinoma cell lines: Hela S3, SiHa and CasKi were purchased from ATCC (Manassas, Va.). Cells were grown routinely in DMEM/HAM F-12K (Sigma, 1:1) containing 10% FBS (Sigma) and 1% Penicillin-streptomycin solution (Sigma, penicillin 10,000 U and streptomycin 10 mg/ml) at 37° C. in 5% CO₂ incubator. The cell line Hep 382.1-7 (Homo sapiens hepatatocellular carcinoma) was purchased from ATCC. It contains an integrated hepatitis B virus genome and was derived from the hepatocellular carcinoma tissue of 8-year old black boy. Cells were grown routinely in Eagle's Minimum Essential Medium (EMEM) (ATCC) containing 10% FBS (Sigma) at 37° C. in 5% CO₂ incubator. Cell layers in 75 ml flask was dispersed by adding 2 ml 1× Trypsin-EDTA solution (Sigma, 0.25% (w/v) trypsin-0.53 mM EDTA) at room temperature for 15 minutes. A2058 cell line derived from a lymph node metastasis from patient with malignant melanoma was obtained from ATCC. The cells were maintained as monolayers in Dulbecco's Modified Eagle's Medium with 4 mM L-Glutamine, 4.5 g/L glucose, 1.5g/L sodium bicarbonate, supplemented with 10% fetal bovine serum and 5% penicillin-streptomycin solution at 37° C. and 5% CO₂, and harvested by using 0.25% (w/v) Trypsin-EDTA solution. Routine maintenance of all cell lines was performed according to the ATCC protocols.

### Western Blots

Cell pellets were suspended in the lysis buffer (4% SDS, 20% glycerol, 0.5 M TrisHCl (pH 6.8), 0.002% bromophenol blue and 10% β-mercaptoethanol). Protein samples were boiled in water for 15 min before running SDS-PAGE. Twenty five-thirty μl protein solution was loaded into each well of 12% precast SDS-PAGE gel (Bio-Rad). SDS-PAGE was used to monitor the relative protein amounts in the samples. Twelve percent SDS-PAGE gel was used to separate proteins, and electrophoresis was performed using Mini-Protean® 3 Cell system (Bio-Rad). After electrophoresis, the gel was transferred into the PVDF transfer buffer (25 mM Tris, 190 mM glycine and 2.5% (v/v) methanol) for 5 min. Then, proteins were transferred from the gel to the Immun-Blot™ PVDF membrane (Bio-Rad) on Semi-dry Electrophoretic Transfer Cell (Bio-Rad) at 15 V for 17 min. The membrane was soaked in the blocking buffer (25 mM TrisHCl (pH7.6), 1 mM EDTA and 150 mM NaCl) for 5 min, and then transferred into the blocking solution (5% non-fat dry milk in the blocking buffer), shaking gently for an hour. The membrane was incubated in TBST solution (0.1% Tween-20, 25 mM TrisHCl (pH7.6) and 500 mM NaCl) containing 1:3000 diluted primary antibody at room temperature for 1 hour with gentle shaking. Then, the membrane was washed with TBST three times (10 min per wash). The membrane was hybridized by the secondary antibody conjugated with alkaline phosphatase (Rabbit polyclonal to mouse IgG H&L (alkaline phosphatase)) in TBST with a 1:100,000 dilution. After three washes with TBST, the membrane was incubated in CDP-Star™ chemiluminescent substrate solution (Sigma) for 5 min, and then exposed to CL-XPosure™ film (Pierce). The film was developed as per manufacturer's instructions.

### MG132 Treatment of Cells

MG132 (Z-LLL-CHO, MW=457.6) purchased from CALBIOCHEM is a potent, reversible and cell-permeable proteasome inhibitor (Ki=4 nM). MG132 was first dissolved in a drop of 100% ethanol followed by DMEM/HAM F-12K medium without addition of FBS, and the stock solution was stored at 4° C. Cell culture was harvested and transferred into 6 sterile test tubes. Each tube contained 0.5-1 ml cell culture (cell concentration was ˜10⁶ cells/rill), and cells were allowed to grow at 37° C. in 5% CO₂ incubator overnight. Then, MG132 solution was added to the tubes for the final concentrations of 0, 1, 2, 5, 10 or 25 μg/ml (0, 2.1, 4.2, 10.5, 21 or 52 μM, respectively). The cells were incubated under the same conditions for another 3 or 6 hours. Finally, cells were harvested by centrifugation and clarified by washing with PBS.

### Radioisotope and Radiolabeling of the Antibodies

The beta-emitter ¹⁸⁸Re with a half life of 16.9 hours was eluted in the form of sodium perrhenate Na¹⁸⁸ReO₄ from an ¹⁸⁸W/¹⁸⁸Re generator (Oak Ridge National Laboratory, Oak Ridge, Tenn.). Antibodies were labeled with ¹⁸⁸Re directly through binding of reduced ¹⁸⁸Re to the generated —SH group on the antibodies as previously described (Kaminski MS et al. (2005) N Engl J Med 352: 441-449.).

### Immunofluorescent Detection of Viral Antigens in Tumor Cells

The tumor cells were grown in the slide chambers at 37° C. for 12 hrs. The medium was removed and the cells were washed with PBS three times. Throughout the procedure, the cells were washed 3 times with PBS after each treatment. They were fixed with 4% paraformaldehyde at room temperature for 20 min followed by permeabilization with 0.3% Triton-X100 in PBS at room temperature for 10 min. Fixed and permeabilized cells were blocked with 5% BSA plus 0.1% Triton X-100 in PBS at room temperature for 30 min. Viral antigens-specific mAbs were diluted 1:200 with the above BSA and Triton X-100 solution and incubated with the cells at room temperature for 60 min. FTIC-conjugated rabbit polyclonal antibody to mouse IgG in 1:300 dilution was added to the cells for 60 min incubation at room temperature in the dark. The slides were viewed with an Olympus AX70 microscope (Melville, N.Y.) equipped with a FITC filter.

### Tumor Models

All animal studies were carried out in accordance with the guidelines of the Institute for Animal Studies at the Albert Einstein College of Medicine. Human cervical carcinoma CasKi and human hepatocellular carcinoma Hep 3B2.1-7 cell lines were chosen as the tumor models based on its expression of E6 and HBx, respectively, and their tumorigenecity in nude mice. Six-week-old female Nu/Nu CD1 nude mice purchased from Charles River were injected into the right flank with 10⁷ CaSki or Hep 382.1-7 cells in 0.1 ml DMEM medium containing 10% fetal calf serum. CasKi tumors began to appear around 60 days after injection, Hep 3B2.1-7 tumors—10 days post cell injection. For biodistribution of HBx-specific mAb, nude mice were inoculated with 10⁷ A2058 human metastatic melanoma cells and 10⁷ Hep 382.1-7 cells into the right and left flanks, respectively. Biodistribution and therapy experiments were initiated when the tumors reached 0.3-0.7 cm in diameter.

### Immunohistochemical Detection of HPV E6 Protein in CasKi Tumors

The tumors taken from CasKi tumor-bearing mice were fixed in 10% buffered formalin overnight, followed by placing in 70% ethanol. The paraffin-embedded tumor tissues were cut into 5 μm slides. The sections were deparaffinized in xylene and dehydrated in graded ethanol continuously, and pre-treated with citrate buffer (pH 6.0) at 100° C. for 20 min to retrieve the antigen. The sections were treated with 3% hydrogen peroxide in methanol in order to block the endogenous peroxidase activity. IHC staining of the tumor tissue sections was then performed using Histostain®-Plus Kits Zymed® 2^{nd} Generation LAB-SA Detection System (Invitrogen) according to the manufacturer's instruction. Mouse mAb C1P5 was used as primary antibody to detect E6 protein in the tumor tissues, and the negative control was incubated with 18B7 mAb instead of primary antibody under the same conditions. Other reagents used in IHC were supplied with the kit.

Western Blot Detection of HBx Antigen in Hep 3B2.1-7 Tumors

The Hep 3B2.1-7 tumors were homogenized on ice and western blot was performed as described above.

### Biodistribution and Scintigraphic Imaging with ¹⁸⁸Re-labeled MAbs.

For biodistribution and scintigraphic imaging in CasKi or Hep 3B2.1-7 tumor-bearing mice, mice were divided into groups of three and injected IP with either 100 μCi of specific mAbs ¹⁸⁸Re-C1P5 or ¹⁸⁸Re-4H9 or with control mAb ¹⁸⁸Re-18B7. Twenty-four hours post-injection mice were anesthetized with Isoflurane and scintigraphically imaged for 2 min on a Siemens gamma camera equipped with ICON image processing software. Forty eight hours post-injection mice were sacrificed and the tumors and muscle were removed, blotted from blood, weighted, counted in a gamma counter and percentage of injected dose per gram tissue and tumor to muscle ratios were calculated.

Therapy of CaSki and Hep 3B2.1-7 tumors in nude mice with ¹⁸⁸Re-labeled mAbs. For therapeutic studies mice with tumors of 0.3-0.7 cm in diameter were randomized into the groups of six. For CasKi tumor-bearing mice, group #1 was treated IP with 350 μCi ¹⁸⁸Re-C1P5 mAb, group #2—with matching amount (30 μg) “cold” C1P5 mAb, and group #3 was left untreated. For RIT of Hep 3B2.1-7 tumor-bearing mice, group #1 was given IP 240 μCi ¹⁸⁸Re-4H9 mAb, group #2—280 μCi, #3—350 #4—500 #5—600 μCi of ¹⁸⁸Re-4H9 mAb, #6—30 μg “cold” 4H9 mAb and group #7 was left untreated. Mice were observed for their well—being and tumor growth. The size of the tumor was measured every 3 days with calipers in three dimensions and the tumor volume was calculated as a product of three dimensions divided by 2. For histological analysis the tumors were removed from the mice at the completion of the experiments, fixed in buffered formalin, parafinized, cut into 5 slices and stained with hematoxylin and eosin (H&E).

Statistical analysis. Non-parametric Wilcoxon Rank Sum test was used to compare organs uptake in biodistribution studies and tumor sizes in therapy studies. The differences were considered statistically significant when P values were <0.05.

### PROPHETIC EXAMPLES

### Prophetic Example 1

### Generation of Anti-Virus Antigen IgG F(ab′)₂ and Fab′ Fragments

Monoclonal IgG antibody 72A1 against Epstein Barr Virus gp 350 has been shown to prevent infection by EBV both in vitro and in mice and humans in vivo (Hague et al. (2006) J. Infect. Dis. 194:584-587). F(ab′)₂ fragments can be obtained by use of a commercial kit (ImmunoPure, Pierce) as in Lendvai et al. (J Infect. Dis. 177: 1647-59, 1998). Briefly, pepsin digestion of IgG at pH 4.2 can be performed, after which the proteolysis is stopped by centrifugation of the pepsin beads and by adjusting pH to 7 with 5 M sodium acetate. Fab′ fragments can be generated by incubation of F(ab′)₂ fragments with 10 mM dithiothreitol followed by 22 mM iodoacetamide to block the thiol groups. The molecular weight of the obtained fragments can be analyzed by sodium dodecyl sulfate-polyacrylamide gel electrophoresis (SDS-PAGE) and by size-exclusion HPLC. The protein concentration can be determined by the method of Lowry et al. (J. Biol. Chem. 193:265-275, 1951) or by other means as are known in the art.

### Prophetic Example 2

### Indirect Labeling of Antibodies and F(ab′)₂ Fragments using Succinimidyl-HYNIC (Hydrazynonicotinamide)

For “indirect” radiolabeling with, e.g., 188-Re and 111-In, antibodies and F(ab′)₂ fragments can be derivatized with, e.g., succinimidyl-HYNIC (hydrazynonicotinamide) and purified as by Blankenberg et al. (J. Nucl. Med. 40:184-191, 1999). The advantage of employing “indirect” labeling via bifunctional chelating agents such as HYNIC or others over “direct” labeling is that the radiolabeling process is greatly simplified and shortened by using the aliquots of HYNIC-binding molecule which can be stored frozen for prolonged periods of time. The incorporation of HYNIC into the proteins can be monitored spectrophotometrically at 385 nm (King et al. (1986) Biochem. 25:5774-5779). The initial HYNIC to protein ratio is preferably chosen so that the final HYNIC to protein ratio does not exceed about 1.5 because above this number a partial loss of immunoreactivity may occur.

### Prophetic Example 3

### Radiolabeling of Binding Molecules by an Attached HEHA

HEHA (1,4,7,10,13,16-hexaazacyclo-hexadecane-N,N′,N′,N′,N′,N′-hexaacetic acid) is a monovalent chelating agent capable of covalent attachment to proteinaceous binding molecules. HEHA is described as a superior carrier for radiation-emitting isotopes for radioimmunotherapy, and is particularly useful with the alpha-emitter 225-Ac (U.S. Pat. No. 6,995,247 to Brechbiel et al.). 225-Ac can be separated from 225-Ra (t_{1/2}=15 days) by ion exchange and extraction column chromatography as described previously (Boll et al. (1997) Radiochim. Acta 79:145-149). Stock solutions of purified 225-Ac in 0.1 M HNO₃ can be freshly prepared as needed. 225-Ac can be complexed with HEHA by mixing approximately 100 microliters of 225-Ac solution (approx. 10 MBq, 0.1 M HNO₃) with 20 microliters of ligand (about 0.01 M in water) and adjusting the pH to near 5.0 by the addition of 5-10 microliters of 1.0 M ammonium acetate. The mixture is kept at 40° C. for 30 minutes and then purified on a Chelex column (Bio-Rad Laboratories, Richmond, Calif.), about 300 microliters bed volume, pre-equilibrated with 0.1 M ammonium acetate, pH=5.0, using 2 ml of ammonium acetate solution as eluant.

Antibody solutions can be initially transferred to dialysis tubing (Spectra/Por CE DispoDialysers, MWCO 10K, 5 ml) and dialyzed against conjugation buffer (1 1, 0.05 M CO₃⁻²/HCO₃⁻¹, 0.15 M NaCl, 5 mM EDTA, pH 8.6) for 6 hours at 4° C. After dialysis, the protein concentration can be determined spectrophotometrically using the Lowry method (Lowry et al., (1951) J. Biol. Chem. 193:265-275) with a bovine serum albumin standard. A solution of HEHA-NCS in water is added to the antibody solution, such that initial molar ratio of ligand to antibody is 50-fold. The reaction mixture is permitted to stand at room temperature overnight (about 18 hours). The reaction mixture is transferred to Centricon (MWCO 30K or 50K) (Amicon) filtration units. Ammonium acetate buffer (0.15 M ammonium acetate, pH 7.0) is added to the filtration unit to a total volume of 1.5 ml and the filtration units are centrifuged until the remaining volume is approximately 0.5 ml. Additional buffer is added and this process is repeated for a total of 6 times. The final antibody concentration can be measured spectrophotometrically and the ligand to protein ratio (L/P) can be determined as previously described (Dadachova et aL, (1999) Nucl. Med. Biol. 26:977 982).

In brief, radiolabeling can be performed using a procedure slightly modified from that described in detail in Mirzadeh et al. (Bioconjugate Chem. 1:59 65, 1990). Labeling is carried out at pH=4-4.2. The pH value of the radiometal solution is adjusted to 3.8-4.0 by addition of several microliters of 3 M NaOAc. The pH of unlabeled HEHA-conjugate is lowered to 4-4.2 by adding the needed amount of 0.15 M ammonium acetate buffer, pH=4.0. To this solution is added the radiometal and the reaction mixture is left at room temperature for 30 minutes. The reaction is quenched by raising the pH to about 6 with 3 M NaOAc and any free radiometal can be scavenged with 5 ml of 0.5 M Na₂EDTA solution. The product can be purified by passage through a TSK-3000 HPLC column (Thompson Instruments) eluted with PBS at 1 ml/minute.

Each of the patents, applications and articles cited herein is incorporated by reference. The foregoing description and the examples are intended as illustrative and are not to be taken as limiting. Still other variations within the spirit and scope of this invention are possible and will readily present themselves to those skilled in the art.

1. A method of treating a subject having a virus-associated cancer comprising administering to the subject a radiolabeled binding molecule, wherein the binding molecule binds to a virus antigen expressed by virus-associated cancer cells in the subject.

2. The method of claim 1, wherein the virus antigen is on the cell surface of virus-associated cancer cells.

3. The method of claim 1, wherein the virus antigen is located intracellularly in virus-associated cancer cells.

4. The method of claim 1, wherein the cancer is a solid tumor, a semi-solid tumor or a liquid tumor.

5. The method of claim 1, any of claims 1 4, wherein the cancer is cervical carcinoma, hepatocellular carcinoma, a lymphoma, Burkitt's lymphoma, nasopharangeal carcinoma, Hodgkin's disease, skin cancer, primary effusion lymphoma, multicentric Castleman's disease, T-cell lymphoma, B-cell lymphoma, splenic lymphoma, adult T-cell leukemia, hary-cell leukemia, Kaposi's sarcoma, post-transplant lymphoma, brain tumor, osteosarcoma, mesothelioma cervical dysplasia, anal cancer, vulvar cancer, vaginal cancer, penile cancer, oropharyneal cancer, nasopharyneal cancer, oral cancer, liver cancer or skin cancer.

6. The method of claim 1, wherein the cancer is cervical cancer.

7. The method of claim 1, wherein the virus is an enveloped virus.

8. The method of claim 1, wherein the virus antigen is a product of a DNA virus.

9. The method of claim 1, wherein the virus antigen is a product of a virus in the family hepadnaviridae, herpesviridae, papillomaviridae, polyomaviridae, or poxviridae.

10. The method of claim 1, wherein the virus antigen is a product of a RNA virus.

11. The method of claim 1, wherein the virus antigen is a product of a virus in the family flaviviridae, paramyxoviridae, retroviridae, or rhabdoviridae.

12. The method of claim 1, wherein the virus antigen is a product of hepatitis B virus (HBV), hepatitis C virus (HCV), Epstein-Barr virus (EBV), human herpes virus 8 (HHV8), a human papilloma virus (HPV), human papilloma virus 16 (HPV16), papilloma virus 18 (HPV18), human papilloma virus 31 (HPV31), papilloma virus 33 (HPV33), papilloma virus 35 (HPV35), papilloma virus 39 (HPV39), papilloma virus 45 (HPV45), papilloma virus 51 (HPV51), papilloma virus 66 (HPV66), simian virus 40 (SV40), JC polyomavirus (JCV), BK polyomavirus (BIN), molluscum contagiosum virus (MCV), mouse mammary leukemia virus (MMLV), human adenoviruses A-F, cytomegalovirus (CMV), human T-cell lymphotrophic virus 1 (HTLV-1), human T-cell lymphotrophic virus 2 (HTLV-2), bovine leukemia virus (BLV), feline leukemia virus (FeLV), Kaposi's sarcoma virus (KSV), Moloney murine sarcoma virus, mouse mammary tumor virus, human immunodeficieny virus-1 (HIV-1), Rous sarcoma virus (RSV), vesicular stomatitis virus (VSV), Newcastle disease virus (NDV), avian erythroblastosis virus, avian myeloblastosis virus, avian carcinoma virus or walleye dermal sarcoma virus (WDSV).

13. The method of claim 1, wherein the virus antigen is a product of a human papilloma virus (HPV).

14. The method of claim 1, wherein the cancer is cervical cancer and wherein the virus antigen is a product of a human papilloma virus (HPV).

15. The method of claim 1, wherein the binding molecule is a paratope-containing molecule.

16. The method of claim 15, wherein the paratope-containing molecule is an intact antibody.

17. The method of claim 16, wherein the antibody is a monoclonal antibody.

18. The method of claim 15, wherein the paratope-containing molecule is a Fab antibody fragment.

19. The method of claim 1, wherein the binding molecule is a single chain polypeptide.

20. The method of claim 1, wherein the radiation-emitting isotope is selected from the group consisting of 11-C, 18-F, 32-P, 34m-Cl, 38-K, 47-Sc, 51-Mn, 52-Mn, 52m-Mn, 52-Fe, 55-Co, 61-Cu, 62-Cu, 64-Cu, 67-Cu, 62-Ga, 67-Ga, 68-Ga, 72-As, 77-As, 75-Br, 76-Br, 82m-Rb, 83-Sr, 87-Sr, 86-Y, 89-Sr, 90-Y, 89-Zr, 94m-Tc, 99m-Tc, 105-Rh, 109-Pd, 111-Ag, 110-In, 111-In, 118-Sb, 120-I, 122-I, 123-I, 124-I, 125-I, 131-I, 177-Lu, 153-Sm, 159-Gd, 166-Ho, 166-Dy, 140-La, 194-Ir, 198-Au, 199-Au, 186-Re, 188-Re, 211-As, 212-Bi, 213-Bi, 212-Pb, 222-Ra, 223-Ra, 224-Ra, 225-Ra, 225-Ac, and 255-Fm.

21. The method of claim 1, wherein the subject is a human.

22. A method of imaging virus-associated tumor cells in a subject comprising administering to the subject a radiolabeled binding molecule, wherein the binding molecule binds to a virus antigen expressed by virus-associated tumor cells in the subject.

23. The method of claim 22, wherein the virus antigen is on the cell surface of virus-associated tumor cells and/or the virus antigen is located intracellularly in virus-associated tumor cells.

24. The method of claim 22, wherein the tumor cells are present in a solid tumor.

25. The method of claim 22, wherein the subject has a cancer selected from the group consisting of cervical carcinoma, hepatocellular carcinoma, a lymphoma, Burkitt's lymphoma, nasopharangeal carcinoma, Hodgkin's disease, skin cancer, primary effusion lymphoma, multicentric Castleman's disease, T-cell lymphoma, B-cell lymphoma, splenic lymphoma, adult T-cell leukemia, hary-cell leukemia, Kaposi's sarcoma, post-transplant lymphoma, brain tumor, osteosarcoma, mesothelioma cervical dysplasia, anal cancer, vulvar cancer, vaginal cancer, penile cancer, oropharyneal cancer, nasopharyneal cancer, oral cancer, liver cancer and skin cancer.

26. The method of claim 22, wherein the virus is an enveloped virus.

27. The method of claim 22, wherein the virus antigen is a product of a DNA virus or a RNA virus.

28. The method of claim 22, wherein the virus antigen is a product of a virus in the family hepadnaviridae, herpesviridae, papillomaviridae, polyomaviridae, poxviridae, flaviviridae, paramyxoviridae, retroviridae or rhabdoviridae.

29. The method of claim 22, wherein the virus antigen is a product of hepatitis B virus (HBV), hepatitis C virus (HCV), Epstein-Barr virus (EBV), human herpes virus 8 (HHV8), a human papilloma virus (HPV), human papilloma virus 16 (HPV16), human papilloma virus 18 (HPV18), human papilloma virus 31 (HPV31), papilloma virus 33 (HPV33), papilloma virus 35 (HPV35), papilloma virus 39 (HPV39), papilloma virus 45 (HPV45), papilloma virus 51 (HPV51), papilloma virus 66 (HPV66), simian virus 40 (SV40), JC polyomavirus (JCV), BK polyomavirus (BKV), molluscum contagiosum virus (MCV), mouse mammary leukemia virus (MMLV), human adenoviruses A-F, cytomegalovirus (CMV), human T-cell lymphotrophic virus 1 (HTLV-1), human T-cell lymphotrophic virus 2 (HTLV-2), bovine leukemia virus (BLV), feline leukemia virus (FeLV), Kaposi's sarcoma virus (KSV), Moloney murine sarcoma virus, mouse mammary tumor virus, human immunodeficieny virus-1 (HIV-1), Rous sarcoma virus (RSV), vesicular stomatitis virus (VSV), Newcastle disease virus (NDV) , avian erythroblastosis virus, avian myeloblastosis virus, avian carcinoma virus or walleye dermal sarcoma virus (WDSV).

30. The method of claim 22, wherein the binding molecule is a paratope-containing molecule.

31. The method of claim 30, wherein the paratope-containing molecule is an intact antibody or a Fab antibody fragment.

32. The method of claim 22, wherein the binding molecule is a monoclonal antibody.

33. The method of claim 22, wherein the binding molecule is a single chain polypeptide.

34. The method of claim 22, wherein the subject is a human.

35. A pharmaceutical composition formulated in dosage form, comprising a radiolabeled binding molecule and a pharmaceutically acceptable carrier, wherein the binding molecule is specific for a virus antigen expressed by a virus-associated cancer cell and wherein the dosage is appropriate to kill cancer cells that express the viral antigen in a subject or wherein the dosage is appropriate to image virus-associated cancer cells in a subject.
